(19) United States
(12) Patent Application Publication
(10) Pub. No.: US 2025/0029301 A1
(43) Pub. Date: Jan. 23, 2025

(54) METHOD AND SYSTEM FOR HOTSPOT CONFIGURATION IN A LEARNING MANAGEMENT SYSTEM

(71) Applicant: D2L Corporation, Kitchener (CA)

(72) Inventors: Kathleen Elizabeth MURPHY, Kitchener (CA); Justin Terrance James SCHLUMKOSKI, Kitchener (CA)

(21) Appl. No.: 18/779,638

(22) Filed: Jul. 22, 2024

Related U.S. Application Data
(60) Provisional application No. 63/514,881, filed on Jul. 21, 2023.

(51) Int. Cl. G06T 11/60; G06T 7/11; G09B 21/00

(52) U.S. Cl. CPC G06T 11/60; G06T 7/11; G09B 21/006; G06T 2207/20104

(57) ABSTRACT

A method and system for hotspot configuration. The method includes: receiving a digital image; receiving one or more hotspot markers for the digital image, wherein the hotspot markers indicate a location of a hotspot in the digital image; preparing a grid to be superimposed over the digital image; displaying the digital image, hotspot markers, and grid; allowing a user to enter alternative text describing the content of the hotspot identified by the hotspot marker; allowing a user to edit the grid, hotspot markers and alternative text; and determining if the grid is valid based on the locations of the hotspot markers within the grid, if not, notifying the user and return to allowing the user to edit; and if so, output the finalized digital image with hotspot markers and alternative text. The system includes a processor, a computer memory, and modules configured to perform the method noted above.

### RELATED APPLICATIONS

The present disclosure claims priority to U.S. Provisional Patent Application No. 63/514,881, filed Jul. 21, 2023, which is hereby incorporated herein in its entirety.

### FIELD

The present disclosure relates generally to hotspot configuration in digital images. More particularly, the present disclosure relates to a method and system for hotspot configuration in digital images in a learning management system.

### BACKGROUND

Learning management systems (“LMS”) are becoming more popular for delivery of educational material in many different situations, whether in conventional areas like public/private educational institutions all the way through to corporations providing internal training to their employees. Some LMSs merely track student registration and progress while others deliver course content and materials directly to students.

With the rapid increase of LMSs and the organizations that use them and provide educational content, there is also an increase in the diversity of learners using such systems, including differently abled learners. Some learners with limited or no visibility make use of screen readers to take part in on-line courses. Some courses make use of digital images to present information. These digital images may typically have an alternative text that can be read by the screen reader to let the learner be aware of the type of digital image displayed. Some digital images will also have interactive icons or locations on the digital image, commonly called hotspots, which can provide further information to the learner when the hotspot is accessed via, for example, a mouse click or mouse over action. The additional information is typically presented in a pop-up window or the like displaying additional content. When using a screen reader, the hotspots may be accessed by pressing a tab key or the like but, depending on the additional content displayed for the hotspot, it may not be accessible by the screen reader.

As such, conventional hotspots are generally not considered sufficiently accessible in that they do not provide a restricted visibility learner with the full or same type of information that a sighted learner will obtain. There is a need for an improved method and system for configuration of hotspots for digital images more generally and in a learning management system, in particular.

The above information is presented as background information only to assist with an understanding of the present disclosure. No determination has been made, and no assertion is made, as to whether any of the above might be applicable as prior art with regard to the present disclosure.

### SUMMARY

In a first aspect, there is provided a method for hotspot configuration, the method including: receiving a digital image; if available, receiving one or more hotspot markers for the digital image, wherein the hotspot markers indicate a location of a hotspot in the digital image; preparing a grid to be superimposed over the digital image; displaying the digital image, hotspot markers, and grid; allowing a user to edit the grid and hotspot markers, including allowing a user to enter alternative text describing the content of the hotspot identified by the hotspot marker; determining if the grid is valid based on the locations of the hotspot markers within the grid, and: if not, notifying the user and return to allowing the user to edit; and if so, outputting the finalized digital image with hotspot markers and alternative text.

In some cases, the preparing a grid may include: automatically creating a default grid dividing the digital image into a predetermined number of portions of the digital image. In this case, the predetermined number of portions may be equally sized or may be proportional to the dimensions of the digital image.

In some cases, the preparing a grid may include: querying a user about the number of rows and columns in the grid and automatically creating a grid based on the number of rows and columns specified by the user. As above, the grid may have a predetermined number of portions that may be equally sized or may be proportional to the dimensions of the digital image.

In some cases, the allowing a user to edit may include: allowing a user to add, move or delete rows and columns in the grid; and allowing a user to add, move or delete hotspot markers and related alternative text. It will be understood that moving rows and columns in the grid may include unequal spacing of grid lines for either rows or columns.

In some cases, the allowing a user to edit further may include allowing a user to edit the digital image itself.

In some cases, the determining if the grid is valid may include determining if there is more than one hotspot marker in a cell of the grid. In some cases, more than one hotspot marker may be allowed in a grid cell and the determining if the grid is valid may include determining if there is more than the allowable number of hotspot markers in a cell of the grid.

According to another aspect herein, there is provided a system for hotspot configuration, the system including: a processor; and a computer memory including computer-readable instructions, which, when executed by the processor, cause the processor to: receive a digital image at a hotspot configuration module; if available, receive one or more hotspot markers for the digital image at the hotspot configuration module, wherein the hotspot markers indicate a location of a hotspot in the digital image; prepare a grid to be superimposed over the digital image via a grid setting module; display the digital image, hotspot markers, and grid via the hotspot configuration module; allow a user to edit the grid and hotspot markers via the hotspot configuration module, including allowing a user to edit alternative text describing the content of the hotspot identified by the hotspot marker; determine if the grid is valid based on the locations of the hotspot markers within the grid at a grid validation module, and: if not, notify the user and return to allow the user to edit; and if so, output the finalized digital image with hotspot markers and alternative text.

In some cases, the prepare a grid may include: automatically creating a default grid dividing the digital image into a predetermined number of portions of the digital image. In this case, the predetermined number of portions may be equally sized.

In some cases, the prepare a grid may include: querying a user about the number of rows and columns in the grid and automatically creating a grid based on the number of rows and columns specified by the user.

In some cases, the allow a user to edit may include: allowing a user to add, move or delete rows and columns in the grid; and allowing a user to add, move or delete hotspot markers and related alternative text.

In some cases, the allow a user to edit further may include allowing a user to edit the digital image itself.

In some cases, the determine if the grid is valid may include determining if there is more than one hotspot marker in a cell of the grid.

Other aspects and features of the present disclosure will become apparent to those ordinarily skilled in the art upon review of the following description of specific embodiments in conjunction with the accompanying figures.

### BRIEF DESCRIPTION OF THE FIGURES

Embodiments of the present disclosure will now be described, by way of example only, with reference to the attached Figures.

FIG. 1 illustrates an example of a learning management system;

FIG. 2 illustrates an example of a computing device for communication with the learning management system of FIG. 1;

FIG. 3 illustrates an example of a hotspot configuration system according to an embodiment herein;

FIG. 4 illustrates an example display of a digital image by a hotspot configuration system according to an embodiment herein;

FIG. 5 illustrates another example display of the digital image of FIG. 4;

FIG. 6 illustrates another example display of the digital image of FIG. 4;

FIG. 7 illustrates another example display of a digital image by a hotspot configuration system according to an embodiment herein;

FIG. 8 illustrates another example display of the digital image of FIG. 7;

FIG. 9 illustrates another example display of the digital image of FIG. 7; and

FIG. 10 is a flowchart illustrating a method for hotspot configuration according to an embodiment herein.

### DETAILED DESCRIPTION

The following description with reference to the accompanying drawings is provided to assist in a comprehensive understanding of example embodiments as defined by the claims and their equivalents. The following description includes various specific details to assist in that understanding but these are to be regarded as merely examples. Accordingly, those of ordinary skill in the art will recognize that various changes and modifications of the embodiments described herein can be made without departing from the scope and spirit of the invention. In addition, descriptions of well-known functions and constructions may be omitted for clarity and conciseness.

The terms and words used in the following description and claims are not intended to be limited to the bibliographical meanings, but are merely used by the inventor to enable a clear and consistent understanding. Accordingly, it should be apparent to those skilled in the art that the following description of embodiments is provided for illustration purpose only and not for the purpose of limiting the invention as defined by the appended claims and their equivalents.

Generally, the present disclosure provides a method and system for configuring hotspots in digital images. In particular, the embodiments of the system and method detailed herein provide for configuring accessible hotspots that are more appropriate for differently abled learners.

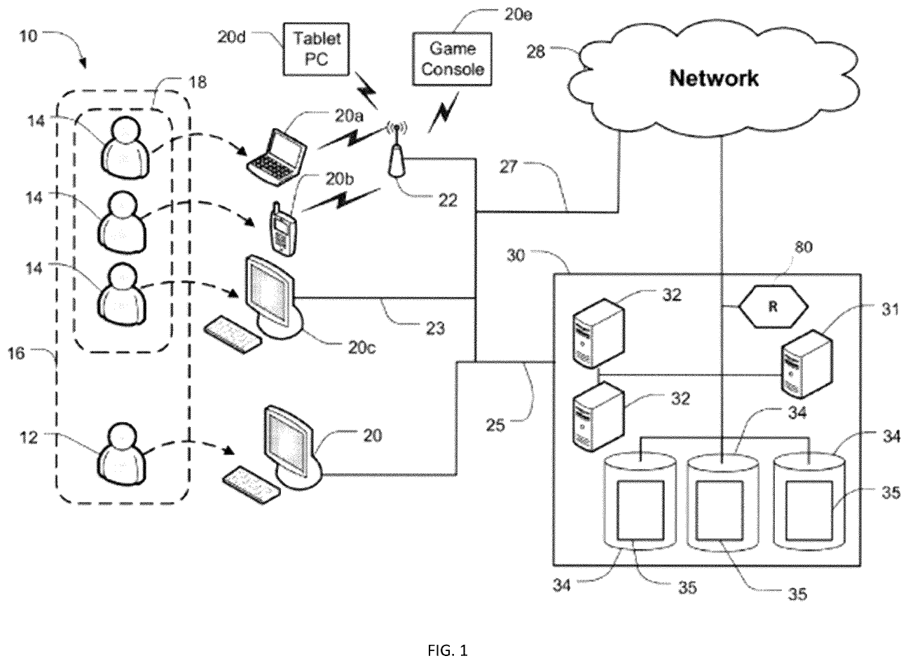

FIG. 1 illustrates an example embodiment of a learning management system 10 according to one embodiment. The learning management system 10 includes an educational service provider system 30, which can be accessed by various users 12, 14 via computer networks.

The users 12, 14 communicate with the educational service provider system 30 either directly or indirectly using any suitable computing device 20, such as, for example a desktop computer that has at least one input device (e.g., a keyboard and a mouse) and at least one output device (e.g., a display screen and speakers). Other examples of the computing device may include: a laptop 20a wirelessly coupled to an access point 22 (e.g., a wireless router, a cellular communications tower, etc.), a wirelessly enabled mobile device, smart phone or the like 20b, a terminal 20c over a wired connection 23, a tablet computer 20d, or a game console 20e over a wireless connection. The computing devices 20 may be connected to the educational service provider system 30 via any suitable communications channel. For example, the computing devices 20 may communicate to the educational service provider system 30 directly through a LAN/intranet or wireless network via a data connection 25, or using an external network, such as, for example, the Internet 28 over a data connection 27.

In some cases, one or more of the users 12 and 14 may be required to authenticate their identities in order to communicate with the educational service provider system 30. For example, the users 12 and 14 may be required to input a login name and/or a password or otherwise identify themselves to gain access to the learning management system 10. In other cases, one or more users (e.g., “guest” users) may be able to access the learning management system 10 without authentication. Such guest users may be provided with limited access, such as the ability to review only one or a few components of the course, for example, to decide whether they would like to enroll in a particular course.

The educational service provider system 30 generally includes a number of components for facilitating the provision of electronic learning services. For example, the educational service provider system 30 generally includes one or more processing devices 32 (e.g., servers), each having one or more processors. The processing devices 32 are configured to send information (e.g., HTML or other data) to be displayed on one or more computing devices 20, 20a, 20b and/or 20c to facilitate social electronic learning (e.g., course information). In some cases, the processing device 32 may itself be a computing device (e.g., a laptop or a personal computer).

The educational service provider system 30 also generally includes one or more data storage devices 34 (e.g., memory, etc.) that are in communication with the processing devices 32, and could include a relational database (such as an SQL database), or other suitable data storage devices. The data storage devices 34 are configured to host data 35 relating to the courses offered by the service provider.

For example, the data 35 can include course frameworks, educational materials to be consumed by the users 14, historical records about assessments or grades of users 14 or assignments completed by the users 14, as well as various other information.

The data storage devices 34 may also store authorization criteria that define which actions may be taken by the users 12 and 14. In some cases, the authorization criteria may include at least one security profile associated with at least one role. For example, one role could be defined for users who are primarily responsible for developing an educational course, teaching it, and assessing work product from students of the course. Users with such a role may have a security profile that allows them to configure various components of the course, to post assignments, to add assessments, to evaluate performance, and so on.

In some cases, some of the authorization criteria may be defined by specific users 40 who may or may not be part of the educational community 16. For example, users 40 may be permitted to administer and/or define global configuration profiles for the learning management system 10, define roles within the learning management system 10, set security profiles associated with the roles, and assign roles to particular users 12, 14 who use the learning management system 10. In some cases, the users 40 may use another computing device (e.g., a desktop computer 42) to accomplish these tasks.

The data storage devices 34 may also be configured to store other information, such as personal information about the users 12, 14 of the learning management system 10, information about which courses the users 12, 14 are enrolled in, roles to which the users 12 and 14 are assigned, particular interests of the users 12,14, and historical information about the performance of the users 12, 14.

The processing devices 32 and data storage devices 34 may also provide other electronic learning management tools (e.g., allowing users to add and drop courses, communicate with other users using chat software, etc.), and/or may be in communication with one or more other vendors that provide various tools.

In some cases, the educational service provider system 30 may also have one or more backup servers 31 that may duplicate some or all of the data 35 stored on the data storage devices 34. The backup servers 31 may be desirable for disaster recovery to prevent undesired data loss in the event of an electrical outage, fire, flood or theft, for example. The backup servers 31 could be located at a remote storage location and the service provider system 30 could connect to the backup server 31 using a secure communications protocol to ensure that the confidentiality of the data 35 is maintained.

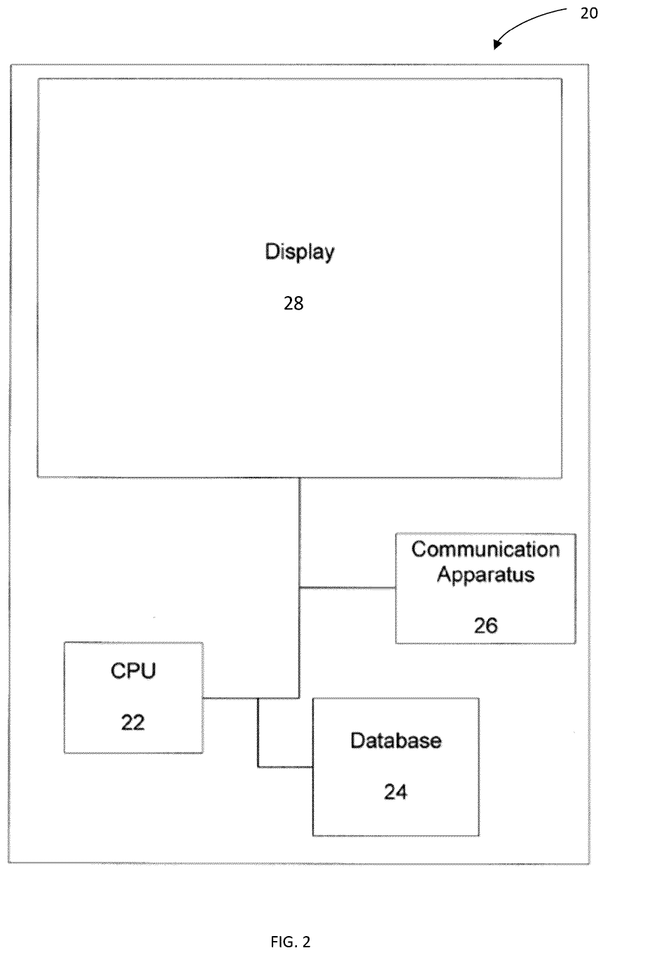

FIG. 2 is a schematic diagram of an example computing device 20, in this case, a mobile computing device, which communicates wirelessly. As shown, the computing device 20 comprises a processor 22, a memory 24, a communication apparatus 26, a display 28, and an input apparatus 29. A user 12, 14, uses the functions of the computing device 20 to interface/communicate with the educational service provider system 30 as described herein.

Generally speaking, the users 12, 14 can use the learning management system 10 to communicate with the educational service provider system 30 to participate in, create, and consume electronic learning services, including enrolling in and participating in various educational courses. In some cases, the educational service provider system 30 may be part of or associated with a traditional “bricks and mortar” educational institution (e.g., an elementary school, a high school, a university or a college), another entity that provides educational services (e.g., an online university, a company that specializes in offering training courses, or an organization that has a training department), or an independent service provider (e.g., for providing individual electronic learning).

It should be understood that a “course” is not necessarily limited to formal courses offered by formal educational institutions. The course may generally include any form of learning instruction offered by an entity of any type. For example, the course may be a training seminar at a company for a small group of employees, a professional certification program with a larger number of intended participants (e.g., PMP, CMA, etc.), and so on.

It should also be understood that users 12, 14 may fall into various categories, including students, instructors, guests, or the like. Further, one or more educational groups can be defined that involve one or more of the users 12, 14. For example, as shown in FIG. 1, the users 12, 14 may be grouped together in an educational group 16 representative of a particular course (e.g., History 101, French 254), in which the first user 12 is an “instructor” and is responsible for providing the course (e.g., organizing lectures, preparing assignments, creating educational content, etc.), while the other users 14 are “learners” or “students” that consume the course content (e.g., the users 14 are enrolled in the course to learn the course content). In some cases, the users 12, 14 may be associated with more than one educational group. For instance, the users 14 may be enrolled in more than one course, while the user 12 may be enrolled in a course and also responsible for teaching a course (which is common for example for graduate students).

In some cases, educational sub-groups may also be defined. For example, in FIG. 1, some users 14 are shown as part of an educational sub-group 18. The sub-group 18 may be defined in relation to a particular project or assignment (e.g., sub-group 18 may be a lab group) or based on other criteria. In some cases, due to the nature of electronic learning, the users 14 in a particular sub-group 18 need not physically meet but may collaborate together using various tools provided by the educational service provider system 30.

In some cases, the groups 16 and sub-groups 18 could include users 12, 14 that share common interests (e.g., interests in a particular sport), that participate in common activities (e.g., users that are members of a choir or a club), and/or have similar attributes (e.g. users that are male, users under twenty-one years of age, etc.).

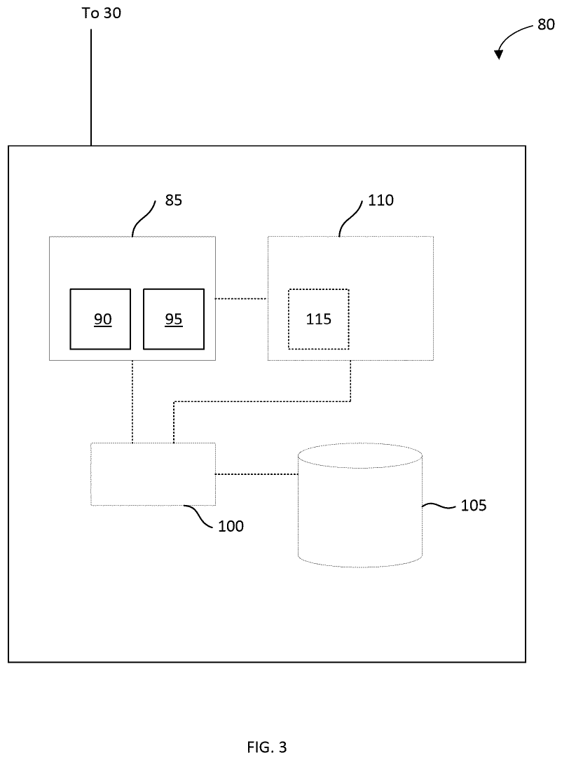

As shown in FIG. 1, the educational service provider system 30 can include a hotspot management system 80. FIG. 3 illustrates a block diagram of the hotspot management system 80 according to one embodiment. The hotspot management system 80 is operable to communicate with a user via the learning management system 10 and a computing device 20. The hotspot management system 80 includes a hotspot configuration module 85, which allows a course creator to configure hotspots for digital images. In this embodiment, the hotspot configuration system 85 includes a grid setting module 90 and a grid validation module 95. The hotspot management system 80 may include its own processor 100 and memory storage/database 105 or may rely on those available through the educational service provider system 30 or otherwise. In some embodiments, the hotspot management system 80 may also include a hotspot presentation module 110 for use by learners accessing the educational service provider system 30. The hotspot presentation module 110 may include a screen reader 115 for reading information to restricted visibility learners (sometimes referred to as “screen reader learners”). It will be understood that alternative software for presenting materials to a learner, including a screen reader, may alternatively be on a user's computer, which may be configured in a similar way as the computer shown in FIG. 2.

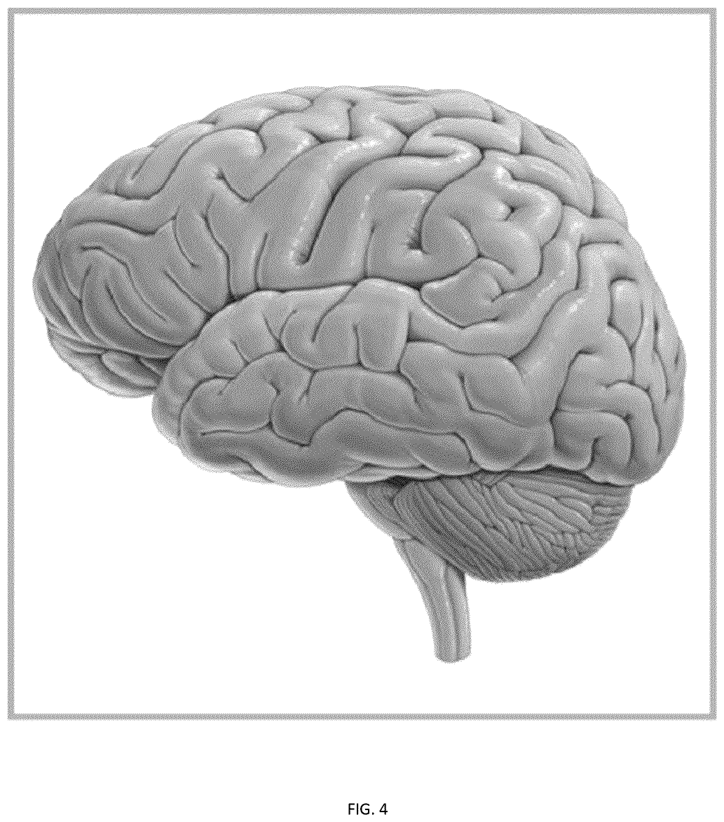

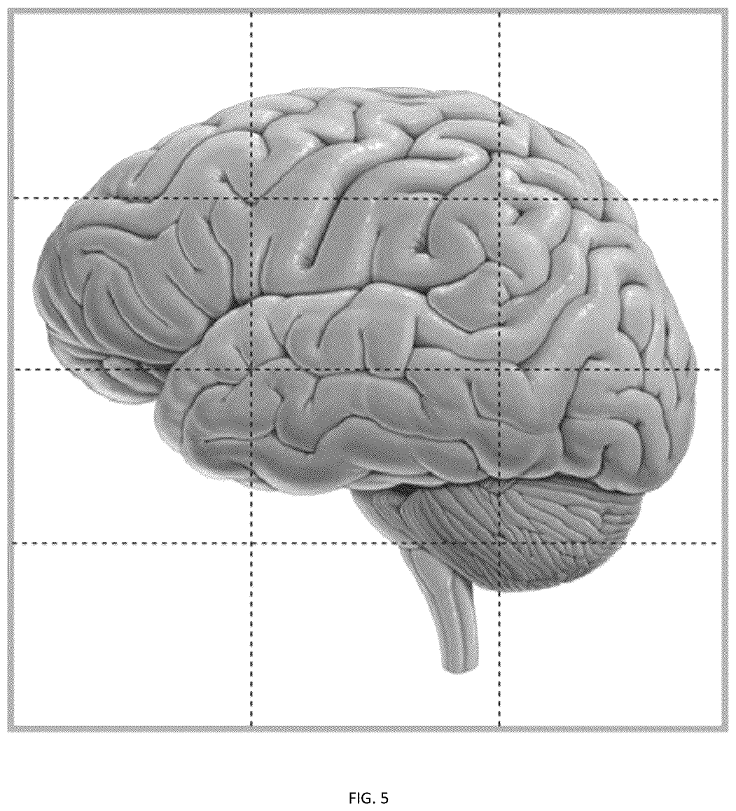

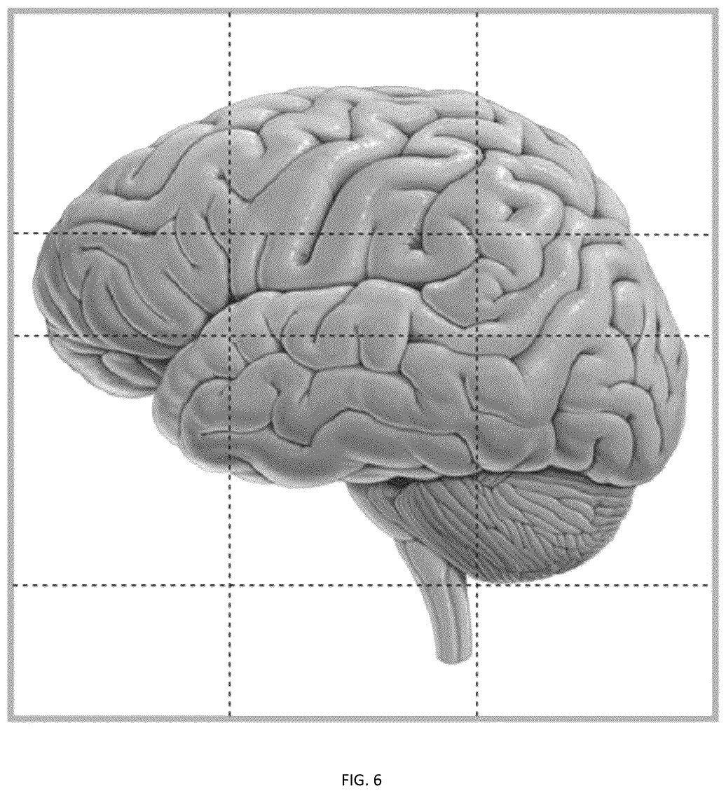

When a user (such as a course or unit creator) desires to include a digital image in a learning module or the like, the hotspot management system 80 and, in particular, the hotspot configuration module 85, can be initiated to allow the user to manipulate the information associated with the digital image. The hotspot configuration module 85 will initially display the digital image as shown in FIG. 4. In some cases, the hotspot configuration module 85, via the grid setting module 90, may query the user as to how many rows and columns to include in the initial grid. In other cases, the grid setting module 90 may create a default grid for display, such as a 2×2 grid or the like, on the digital image as shown in FIG. 5. In this case, the default grid can be configured to divide the digital image into an equal number of portions or the like. The grid will include a number of cells identified by, for example, a row and column number such as row 1, column 3, or the like. Once the digital image and initial grid are displayed, the grid setting module 90, allows the user to adjust the grid by moving grid lines, as shown in FIG. 6. At this point, the digital image may already have hotspot markers included (not shown in FIGS. 4-6), such as those already provided with the digital image, which will appear as icons on the digital image, or alternatively, the system may allow a user to add hotspot markers to the digital image. In either event, the grid validation module 95 reviews the hotspot markers to ensure that there is only one hotspot marker per grid cell/location (row/column combination) and if there is more than one hotspot marker in a grid cell, notifies the user. In some cases, as an alternative, the hotspot configuration module 85 may automatically attempt to adjust the position of one or more grid lines to attempt to resolve the invalidity issue and then notify the user of the change.

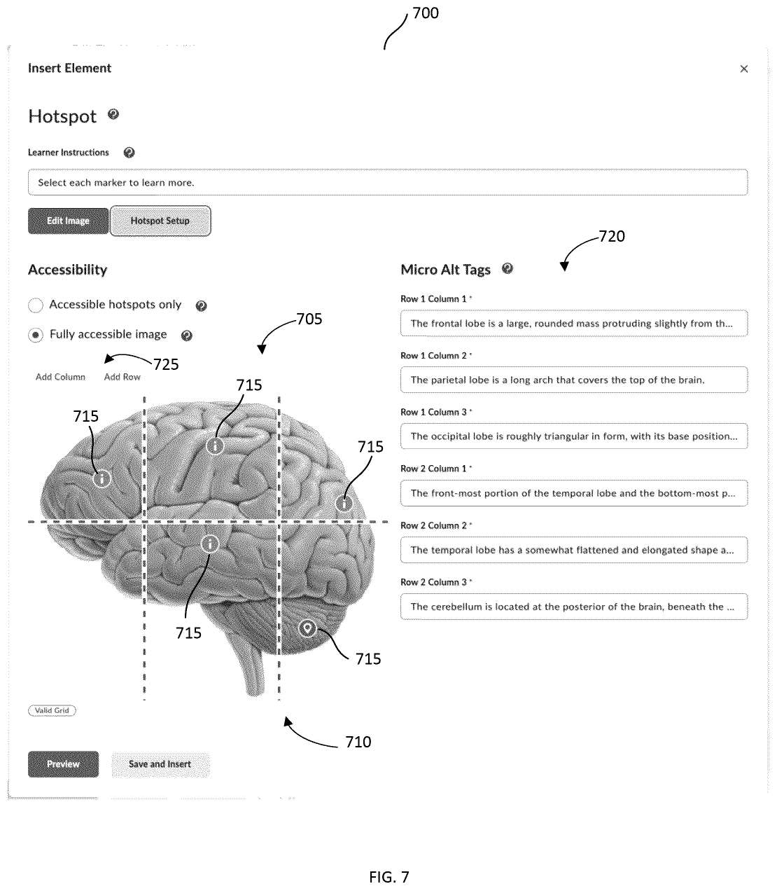

FIGS. 7-9 illustrate example display screens 700 used by an embodiment of the hotspot configuration module 85. In FIG. 7, a user is presented with the digital image 705 and an initial grid 710 as well as hotspot markers 715 (identified by icons) showing the location of hotspots on the image. While FIGS. 7-9 do not show the content associated with the hotspots (for standard learners), FIGS. 7-9 do illustrate data entry locations for alternative text entries 720 that can be used to describe the content associated with the hotspots (in this case, identified as “micro alt Tags™”). The alternative text entries 720 are somewhat similar to the alternative text that will typically accompany the digital image itself in order to provide information about the content of the image. The alternative text entries are related to each of the row/column combinations within the grid, and thereby, to a respective hotspot or hotspot marker in a grid location. In FIG. 7, the user may have already indicated the locations of hotspots within the digital image (via the hotspot markers) or added the hotspot markers. As noted herein, hotspots can add further information about a portion of the digital image or the like. Hotspot markers can use different icons or colors of icons, depending on the type of content in the hotspot, to provide additional support for sighted users, or the like.

FIGS. 7-9 also illustrate options with regard to configuring the structure of the digital image and hotspots. For example, there are options for: “Accessible hotspots only” and “Fully accessible image”. The term “Accessible hotspots only” refers to a digital image and hotspots that allows the user to add a micro alt tag to describe the content of each hotspot. Then, for a screen reader learner, the experience would be to access the digital image by selecting/“tabbing” into the digital image to hear the alt tag for the full image then to use the arrow keys to navigate to each hotspot in the order the configuration/creator user input the hotspots and hear the micro alt tag for the hotspot. This may be an appropriate option for when the context surrounding each hotspot or the spatial relationship between hotspots isn't relevant to the learning (for example, when an image is being used to demonstrate steps in a process, when the image is an infographic, or the like). The term “fully accessible image” makes use of the grid to allow users to add a micro alt tag to one or more cells in the grid. With this option, the screen reader learner tabs into the digital image to hear the alt tag for the full image (for example, “Illustrated side view of the human brain floating in white space. The front of the brain faces left. The image includes a portion of the brain stem”). Next, the arrow keys can be used to invoke the accessible grid that the screen reader learner can navigate cell by cell, receiving information about each cell's position within the grid (for example, Row 1, Column 1) as well as alternative text information about the contents of the portion of the digital image in the grid cell or about the content of a hotspot within the grid cell (via the micro alt tags, for example, “The frontal lobe is a large, rounded mass protruding slightly from the rest of the brain”). In this case, the grid can also provide additional context about the digital image that could be missing otherwise if only providing information about the content of the hotspots. For example, in FIGS. 7-9, there is no specific hotspot on the brain stem, however, the micro alt tags can help to indicate the existence and position of this brain part in relation to the brain lobes that are the focal point of the activity. The option of using the grid provides screen reader learners with spatial information about the hotspots allowing them an experience more similar to sighted learners.

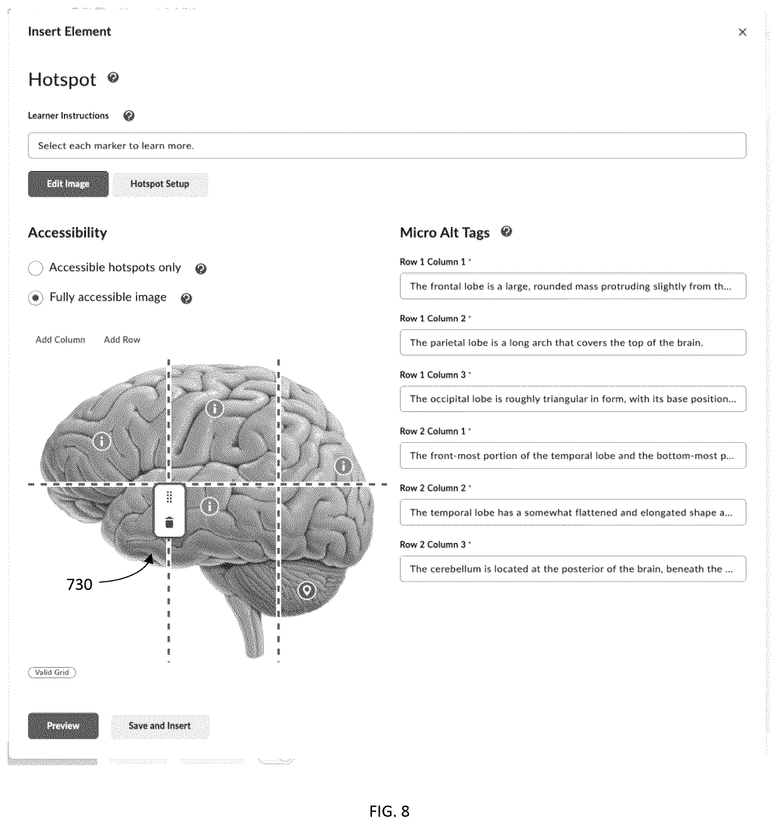

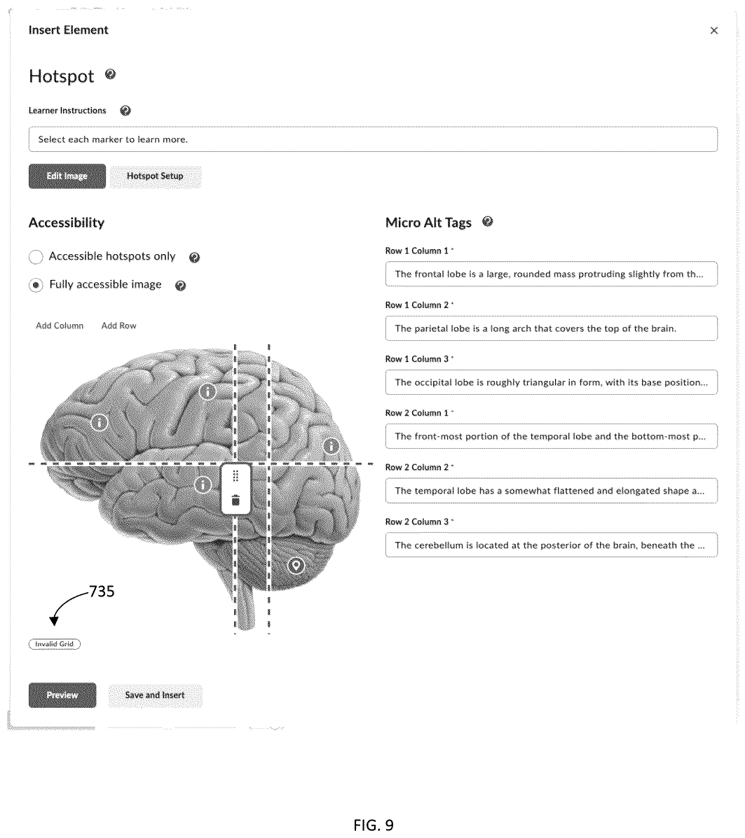

As shown in FIGS. 7-9, a user can select to add an additional column or row to the grid 725. FIG. 8 illustrates that when a user selects a column or row, a “handle” icon 730 is provided to allow the grid line to be adjusted or deleted. FIG. 9 illustrates that the hotspot configuration module 85 monitors for and detects situations in which more than one hotspot marker is within a single grid (row, column) location and indicates “invalid grid” 735 in this situation. This prevents multiple hotspots within one grid location. Although there may be some cases where there could be more than one hotspot, for example, an A and B hotspot within one grid location, however, this may not provide the same benefit. However, particularly in larger images, there may be a situation in which a nested grid may be provided within a cell of an original grid.

In FIGS. 7-9, the micro alt tag entry boxes will expand while editing to allow a user to view/edit the entire alternative text describing the hotspot content for a particular grid location. When configuring, the user may also be able to edit/replace the digital image, grid, edit/reposition hotspots markers; change hotspot icon and/or color, or the like. When a digital image is changed, a user may be provided an option to either save or remove existing hotspot markers, grid, and tags, or the like.

Once the digital image and hotspots has been configured by the hotspot configuration module 85, a restricted visibility learner (screen reader learner) can access the digital image (with hotspots) via the hotspot presentation module 110 and the screen reader 115. As noted above, the screen reader can read the alternative text entries (micro alt tags) for the hotspots, in some cases, prefaced by the location of the hotspot on the grid. For example, the screen reader will read “Row 1, Column 1” and then the related alternative text entry, then “Row 1, Column 2” and the related alternative text entry, and so forth until the digital image and hotspots have been covered. As noted herein, in some cases, there can even be an alternative text entry for a grid location/cell that may not have been assigned a hotspot for sighted learners. Without a grid system, there is a risk that the screen reader would read the alternative text entries for the content associated with the hotspot without any indication as to the location on the digital image or in a way that could confuse the learner about the location of the hotspots on the digital image. In this way, the use of a grid provides additional information to screen reader learners. When presenting the digital image, the digital image may be displayed without the grid lines until a screen reader learner accesses the grid by “tabbing” into the image and/or by using the arrow keys, or the like, to access the hotspots/grid cells within the digital image.

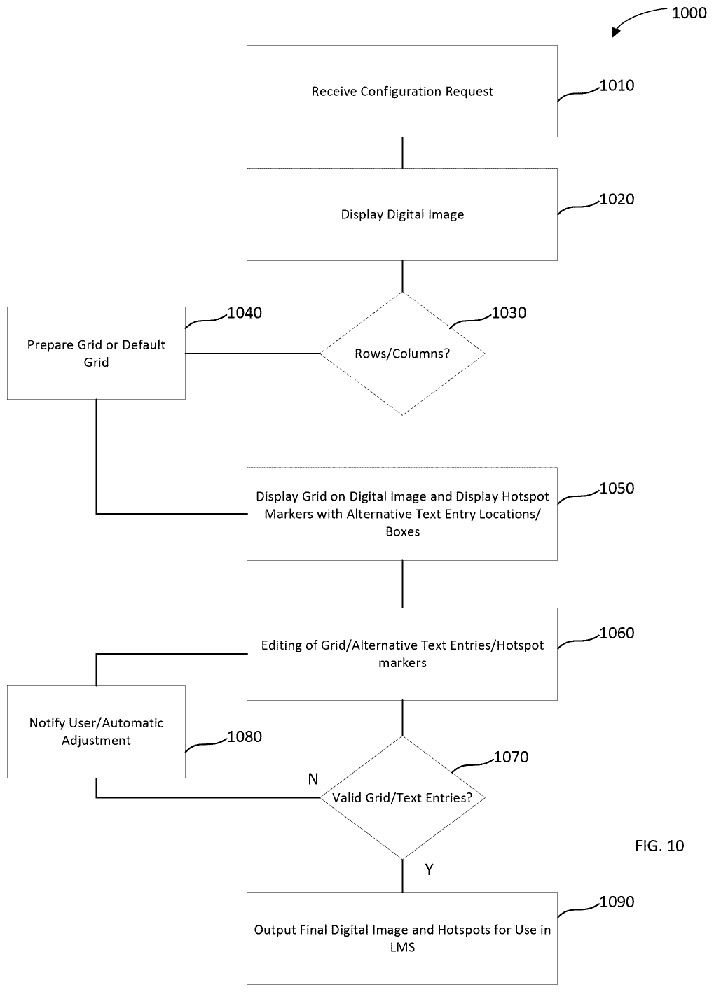

FIG. 10 is a flowchart illustrating a method 1000 for hotspot configuration according to an embodiment. The method starts when the hotspot configuration module 85 receives a request to begin configuration at 1010. The hotspot configuration module 85 then displays the digital image to be configured with hotspots at 1020. The hotspot configuration module 85 may query a user for the number of rows and columns in an initial grid at 1030. If the hotspot configuration module 85 does not request the rows/columns, it may alternatively present a default grid. In either case, the hotspot configuration module 85 prepares a grid at 1040 to be superimposed on the digital image at 1050. At 1050, the hotspot configuration module 85 also displays grid locations and alternative text entry boxes associated with each of the grid locations In some cases, the hotspot markers may be pre-placed on the digital image at the time the digital image is displayed (i.e. before adding the grid) while, in other cases, the hotspot markers may be added while configuring the grid. The hotspot configuration module 85 then, at 1060, allows the user to edit the digital image and grid by adding, removing or moving grid bars (i.e. rows and columns), the hotspot markers (location, icon, color, or the like), and the alternative text entries associated with each hotspot and/or grid location. The hotspot configuration module 85 then determines if the grid is valid based on the locations of hotspot markers within the grid locations at 1070. If the grid is invalid due to more than one hotspot per grid location or the like, the hotspot configuration module 85 can attempt to automatically adjust the grid and/or notify the user, at 1080, and returns to editing. Otherwise, the hotspot configuration module 85 outputs the final digital image with hotspots and hotspot markers at 1090 for use in the LMS.

In the preceding description, for purposes of explanation, numerous details are set forth in order to provide a thorough understanding of the embodiments. However, it will be apparent to one skilled in the art that these specific details may not be required. In other instances, structures may be shown in block diagram form in order not to obscure the understanding. For example, specific details are not provided as to whether the embodiments described herein are implemented as a software routine, hardware circuit, firmware, or a combination thereof.

Embodiments of the disclosure can be represented as a computer program product stored in a machine-readable medium (also referred to as a computer-readable medium, a processor-readable medium, or a computer usable medium having a computer-readable program code embodied therein). The machine-readable medium can be any suitable tangible, non-transitory medium, including magnetic, optical, or electrical storage medium including a diskette, compact disk read only memory (CD-ROM), memory device (volatile or non-volatile), or similar storage mechanism. The machine-readable medium can contain various sets of instructions, code sequences, configuration information, or other data, which, when executed, cause a processor to perform steps in a method according to an embodiment of the disclosure. Those of ordinary skill in the art will appreciate that other instructions and operations necessary to implement the described implementations can also be stored on the machine-readable medium. The instructions stored on the machine-readable medium can be executed by a processor or other suitable processing device and can interface with circuitry to perform the described tasks.

The above-described embodiments are intended to be examples only. Alterations, modifications and variations can be effected to the particular embodiments by those of skill in the art without departing from the scope, which is defined solely by the claims appended hereto.

What is claimed is:

1. A method for hotspot configuration, the method comprising:
receiving a digital image;
if available, receiving one or more hotspot markers for the digital image, wherein the hotspot markers indicate a location of a hotspot in the digital image;
preparing a grid to be superimposed over the digital image;
displaying the digital image, hotspot markers, and grid;
allowing a user to edit the grid and hotspot markers, including allowing a user to enter alternative text describing the content of the hotspot identified by the hotspot marker;
determining if the grid is valid based on the locations of the hotspot markers within the grid, and:
if not, notifying the user and return to allowing the user to edit; and
if so, outputting the finalized digital image with hotspot markers and alternative text.

2. A method according to claim 1, wherein the preparing a grid comprises:
automatically creating a default grid dividing the digital image into a predetermined number of portions of the digital image.

3. A method according to claim 2, wherein the predetermined number of portions are equally sized.

4. A method according to claim 1, wherein the preparing a grid comprises:
querying a user about the number of rows and columns in the grid and automatically creating a grid based on the number of rows and columns specified by the user.

5. A method according to claim 1, wherein the allowing a user to edit comprises:
allowing a user to add, move or delete rows and columns in the grid; and
allowing a user to add, move or delete hotspot markers and related alternative text.

6. A method according to claim 1, wherein the allowing a user to edit further comprises allowing a user to edit the digital image itself.

7. A method according to claim 1, wherein the determining if the grid is valid comprises determining if there is more than one hotspot marker in a cell of the grid.

8. A system for hotspot configuration, the system comprising:
a processor; and
a computer memory including computer-readable instructions, which, when executed by the processor, cause the processor to:
receive a digital image at a hotspot configuration module;
if available, receive one or more hotspot markers for the digital image at the hotspot configuration module, wherein the hotspot markers indicate a location of a hotspot in the digital image;
prepare a grid to be superimposed over the digital image via a grid setting module;
display the digital image, hotspot markers, and grid via the hotspot configuration module;
allow a user to edit the grid and hotspot markers via the hotspot configuration module, including allowing a user to edit alternative text describing the content of the hotspot identified by the hotspot marker;
determine if the grid is valid based on the locations of the hotspot markers within the grid at a grid validation module, and:
if not, notify the user and return to allow the user to edit; and
if so, output the finalized digital image with hotspot markers and alternative text.

9. A system according to claim 8, wherein the prepare a grid comprises:
automatically creating a default grid dividing the digital image into a predetermined number of portions of the digital image.

10. A system according to claim 9, wherein the predetermined number of portions are equally sized.

11. A system according to claim 8, wherein the prepare a grid comprises:
querying a user about the number of rows and columns in the grid and automatically creating a grid based on the number of rows and columns specified by the user.

12. A system according to claim 8, wherein the allow a user to edit comprises:
allowing a user to add, move or delete rows and columns in the grid; and
allowing a user to add, move or delete hotspot markers and related alternative text.

13. A system according to claim 8, wherein the allow a user to edit further comprises allowing a user to edit the digital image itself.

14. A system according to claim 8, wherein the determine if the grid is valid comprises determining if there is more than one hotspot marker in a cell of the grid.
